(19) United States
(12) Patent Application Publication
(10) Pub. No.: US 2008/0286785 A1
(43) Pub. Date: Nov. 20, 2008

(54) Method to predict or monitor the response of a patient to an erbb receptor drug

(75) Inventors: Kazuto Nishio, Tokyo (JP); Hideharu Kimura, Tokyo (JP); Kazuo Kasahara, Kanazawa (JP)

Correspondence Address: Edwards Angell Palmer & Dodge LLP, P.O. Box 55874, Boston, MA, 02205, US

(73) Assignee: AstraZeneca UK Limited, London (GB)

(21) Appl. No.: 12/080,959

(22) Filed: Apr. 7, 2008

Related U.S. Application Data
(63) Continuation of application No. PCT/GB05/04036, filed on Oct. 20, 2005.

(30) Foreign Application Priority Data
Oct. 5, 2005 (GB) PCT/GB2005/003823

(51) Int. Cl. C12Q 1/68

(52) U.S. Cl. 435/6

(57) ABSTRACT

The invention provides a method of detecting ErbB receptor mutations comprising the steps of providing a bio-fluid sample from a patient; extracting DNA from said sample; and screening said DNA for the presence of one or more mutations that alter tyrosine kinase activity in the receptor.

### RELATED APPLICATIONS

This is a continuation patent application that claims priority to PCT patent application number PCT/GB2005/004036, filed on Oct. 20, 2005, which claims priority to PCT/GB2005/03823 filed on Oct. 5, 2005, the entirety of which are herein incorporated by reference.

### FILED OF INVENTION

The present invention relates to a method for predicting or monitoring the response of a patient to an ErbB receptor drug, for example gefitinib, which targets the epidermal growth factor receptor (EGFR). The method provides a sensitive and specific screen for mutations in genomic DNA occuring at low concentrations in bio-fluids such as serum. the method is suitable for detecting mutations that are known to increase ErbB tyrosine kinase receptor activity and appear to correlate with a response to ErbB receptor drug treatment.

ErbB receptors are protein tyrosine kinases (TKs) belonging to the TK superfamily, the members of which a regulate signaling pathways controlling growth and survival of cells. The ErbB family of receptors consists of four closely related subtypes: ErbB1 (epidermal growth factor receptor [EGFR]), ErbB2 (HER2/neu), ErbB3(HER3), and ErbB4)HER$) (Cell. 2000; 103:211-255).

Signaling from the EGFR for example, is triggered by the binding of growth factors such as epidermal growth factor (EGF), resulting in the dimerization of EGFR molecules or heterodimerization with other closely related receptors such as HER2/neu. Autophosphorylation and trasnphosphorylation of the receptors through their tyrosine kinase domains leads to the recruitment of downstream effectors and the activation of proliferative and cell-survival signals (Exp. Cell. Res. 2003; 284:31-53. When overexpressed or activated by mutations, ErbB receptor TKs can lead to the development of breast cancer, non-small-cell lung caner (NSCLC), colorectal cancer, head and neck cancer, and many other solid tumours (Exp. Cell. Res. 2003; 284:122-130). EGRF is overexpressed in 40 to 80 percent of non-small cell lung caners and many other epithelial cancers (N. Engl. J. Med. 2004; 350(21):2129-2139). Anticancer therapy has been designed to target the products of such genes in order to inhibit their activity. The drug gefitinib for example, is a potent inhibitor of the EGFR family of tyrosine kinase enzymes such as ErbB1 and was approved in Japan on Jul. 5, 2002 for treatment of inoperable or recurrent NSCLC.

Patents vary in their responses to any prescribed medications, both with respect to how well it works (its efficacy) and adverse reactions to it (side effects). In the case of gefitinib, patients exhibit a differential response to the tyrosine kinase inhibitor treatment including a group of about 10 percent of patients that have a rapid and often dramatic clinical response (N. Engl. J. Med.2004; 350(21):2129-2139). Accordingly there is a need to identify pre-treatment those patients who will respond to the drug and also to identify post treatment those patients that are responding to the drug, so that the medicine can be targeted more effectively.

It has recently been discovered that a subgroup of patients with non-small cell lung cancer has specific mutations in the EGFR gene which appear to correlate with clinical responsiveness to the tyrosine kinase inhibitor gefitinib (Science 2004; 304:1497-1500). These mutations lead to increased growth factor signalling and confer susceptibility to the inhibitor. It is thought that screening for such mutations in lung cancers may identify patients who will have a response to gefitinib (J. Clin. Oncol. 23; 2493-2501). However, to date, the only way that mutations can be measured reliably is by analysis of solid tissue samples by taking a tumour biopsy from the patient. This is a difficult procedure, is very unpleasant for the patient and sometimes impossible when a tumour is inoperable.

Another problem in screening patients for mutations is the difficulty in detecting mutant genes among an excess of wild-type genes. This is a known problem in the art and especially important given that identification of mutant DNA at low concentration could be critical for early detection of a tumour or to identify the appropriate course of treatment for a patient at an early stage (Clin Cancer Res. 2004; 10(7):2379-85). Accordingly, there is a need for less invasive and more reliable ways to monitor and predict the response of patients to ErbB receptor drugs, for example before embarking them on a therapy that may be very effective, but for only a small percentage of those patients.

### SUMMARY OF THE INVENTION

We have found a method of reliably detecting ErbB receptor mutations in bio-fluid samples taken from patients, that can be used to predict a patients' response or survival benefit from an ErbB receptor drug. In particular, the presence of a mutation that alters the tyrosine kinase activity of an ErbB receptor indicates that a patient may respond positively to the drug whilst the presence of only the wild type allele indicates that the patient may not respond to an ErbB receptor drug.

According to the first aspect of the invention there is provided a method for detecting ErbB mutations comprising the steps of:
(a) providing a bio-fluid sample from a patient
(b) extracting DNA from said sample; and
(c) screening said DNA for the presence of one or more mutations in the receptor.

Preferably the method for detecting ErbB mutations described above comprises detection of one or more mutations in an ErbB receptor that alter the tyrosine kinase activity in said receptor.

Most preferably the ErbB receptor in the above described method is EGFR.

The present inventors have found that measurement of mutations in bio-fluid samples in patients may be used both to predict and to monitor the effects of ErbB receptor drugs in vivo.

In a preferred aspect, the invention provides a method for predicting the response of a patient to an ErbB receptor drug comprising the steps of:
(a) providing a bio-fluid sample from a patient
(b) extracting DNA from said sample
(c) screening said DNA for the presence of one or more mutations that alter tyrosine kinase activity in the receptor

In another embodiment of the invention there is provided a method for monitoring the response of a patient to an ErbB receptor drug comprising the steps of:
(a) providing a bio-fluid sample from a patient
(b) extracting DNA from said sample
(c) screening said DNA for the presence of one or more mutations that alter tyrosine kinase activity in the receptor.

As will be understood by those skilled in the art, monitoring of a response to an ErbB receptor drug allows the response of a patient to whom the drug has already been administered to be assessed; thus, it is applied to patients post-treatment. However, prediction of a response is carried out in patents not exposed to an ErbB receptor drug, and is carried out pre-treatment.

In another embodiment the method comprises the steps described above wherein the prediction of the response of a cancer patient to an ErbB receptor drug predicts the survival benefit to the patient.

Preferably a method of predicting a response to an ErbB drug as described above further comprises the step of:
(d) concluding that patients in which both mutated and wildtype alleles are detected will respond positively to an ErbB receptor drug, whereas patients in which only wild type alleles are detected will not respond positively to the drug.

In another embodiment the method of screening described above comprises use of polymerase chain reaction with allele specific primers that detect single base mutations, small in-frame deletions or base substitutions.

Preferably the method of screening involves use of real time polymerase chain reaction (real time-PCR) with allele specific primers that detect single base mutations, small in-frame deletions or base substitutions.

In a further embodiment the method of predicting a response to an ErbB drug is as described above wherein a first primer pair is used to detect the wild type allele and a second primer pair is used to detect the mutant allele; and wherein one primer of each pair comprises:
(a) a primer with a terminal 3′ nucleotide that is allele specific for a particular mutation; and
(b) possible additional mismatches at the 3′ end of the primer.

Preferably, one primer in each pair as described above further comprises:
(a) a single molecule or nucleic acid duplex probe containing both a primer sequence and a further sequence specific for the target sequence;
(b) a fluorescent reporter dye attached to the 5′ end of the probe in close proximity with a quencher molecule within said single molecule or nucleic acid duplex;
(c) one or more non-coding nucleotide residues at one end of said probe;
(d) wherein said reporter dye and quencher molecule become separated during amplification of the target sequence.

Advantageously, the probe is a Scorpion® probe.

Preferably the method according to the invention uses a technique capable of detecting a mutant sequence present at 10% of the level of wild type sequence. More preferably the technique can detect mutant sequence at 9%, 8%, 7%, 6%, 5%, 4%, 3%, 2%, 1%, 0.1% or 0.01% of the levels of the wild type sequence.

The fluorescent probe system described above has the advantage that no separate probe is required to bind to the amplified target, making detection both faster and more efficient than other systems. The present invention demonstrates that the use of Scorpion® primers in an ARMS amplification system enhances the sensitivity of methods used to detect EGFR mutations (See Example 4).

Preferably the bio-fluid described in the method above is any one of blood, serum, plasma, sweat or saliva. Advantageously, the bio-fluid is serum.

Most previous studies looking at the correlation between EGFR mutations and NSCLC progression demonstrated such mutations in operative resected tumour samples taken after commencement of treatment, for a retrospective study. However the difficulty in sampling inoperable NSCLC tumours from patients at an earlier stage has hampered attempts to perform prospective studies with the potential to select patients before the commencement of treatment.

However, the present invention provides a method of detecting mutant EGFR from cancer patients' samples other than tumour specimens. The sampling of bio-fluids is less invasive than previous methods of analysing EGFR mutations in cancer patients. In contrast to collection of tumour samples, serum samples for example, can be collected easily and tests can be repeated. Furthermore, tumour cells are known to release DNA into the circulation, which is enriched in the serum and plasma, allowing detection of mutations and microsatellite alterations in the serum DNA of cancer patients (Cancer Res. 1999; 59(1):67-70).

In a further embodiment of the invention, the ErbB receptor drug is an ErbB receptor tyrosine kinase inhibitor. Preferably the ErbB receptor drug is an EGFR tyrosine kinase inhibitor. In a preferred method, the EGFR tyrosine kinase inhibitor is selected from a group consisting of gefitinib, erlotinib (Tarceva, OSI-774, CP-358774), PKI-166, EKB-569, HKI-272 (WAY-177820), lapatinib (GW2016, GW-572016, GSK572016), canertinib (CI-1033, PD183805), AEE788, XL647, BMS 5599626, ZD6474 (Zactima™) or any of the compounds as disclosed in WO2004/006846 or WO2003/082290.

In another embodiment of the invention the ErbB receptor drug is an EGFR inhibitor. Preferably, the EGFR inhibitor is an anti-EGFR antibody selected from the group consisting of cetuximab (Erbitux, C225), matuzumab (EMD-72000), panitumumab (ABX-EGF/rHuMAb-EGFR), MR1-1, IMC-11F8 or EGFRL11.

Preferably, the method of any preceding claim comprises an ErbB receptor drug used as monotherapy or in combination with other drugs.

In a most preferred embodiment, the EFGR tyrosine kinase inhibitor drug is selected from a group consisting of gefitinib, erlotinib (Tarceva, OSI-774, CP-358774), PKI-166, EKB-569, HKI-272 (WAY-177820), lapatinib (GW2016, GW-572016, GSK572016), canertinib (CI-1033, PD183805), AEE788, XL647, BMS 5599626, ZD6474 (Zactima™) or any of the compounds as disclosed in WO2004/006846 or WO2003/082290.

The mutations in the invention are found to occur as insertions, deletions or substitutions of nucleic acid. The mutations preferably occur in the tyrosine kinase domain of an ErbB receptor. Preferably the mutations occur in the tyrosine kinase domain of EGFR. Preferably, the mutations are selected from the group of EGFR mutations listed in Table 5. Advantageously the mutations cluster around the ATP binding site in exons 18, 19, 20 or 21 of EGFR. Preferably the mutations are selected from the group of EGFR mutations listed in Table 5. In a most preferred embodiment, the mutations are E746_A750del in exon 19 and L858R in exon 21 of EGFR.

Approximately 30 mutations in exon 18-21 of EGFR have been detected in lung tumour specimens. Among the NSCLC-associated EGFR mutations detected in tumour specimens, the 15-bp nucleotide in-frame deletions in exon 19 (E746_A750del) and the point mutation which is a replacement of leucine by arginine at codon 858 in exon 21 (L858R) account for approximately 90% of these mutations (Cancer Res. 2004; 64:8919-8923, Proc. Natl. Acad. Sci USA 2004; 101:13306-13311).

Advantageously the patient suffers from a cancer selected from the group consisting of non-solid tumours such as leukaemia, multiple myeloma or lymphoma, and also solid tumours, for example bile duct, bone, bladder, brain/CNS, glioblastoma, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval tumours.

In another embodiment of the invention, the method as described above further comprises the step of:
(e) screening said DNA for the presence of one or more mutations in components of the downstream signalling pathway of an ErbB receptor.

A second aspect of the invention encompasses a composition comprising a first primer pair which is used to detect the wild type allele and a second primer pair which is used to detect the mutant allele of an ErbB receptor wherein one primer of each pair further comprises:
(a) a primer with a terminal 3′ nucleotide that is allele specific for a particular mutation; and
(b) possible additional mismatches at the 3′ end of the primer.
(c) a single molecule or nucleic acid duplex probe containing both a primer sequence and a further sequence specific for the target sequence;
(d) a fluorescent reporter dye attached to the 5′ end in close proximity with a quencher molecule within said single molecule or nucleic acid duplex;
(e) one or more non-coding nucleotide residues at one end of said probe;
(f) wherein said reporter dye and quencher molecule become separated during amplification of the target sequence.

A third aspect of the invention comprises use of a primer specific for ErbB receptor in an assay conducted in a bio-fluid for predicting the response of a patient to an ErbB drug.

Preferably the use described above includes manufacture of a composition for testing a bio-fluid for predicting the response of a patient to an ErbB drug.

Advantageously, the above-described use further comprises the steps of:
(a) extracting DNA from said sample
(b) screening said DNA for the presence of one or more mutations that alter tyrosine kinase activity in the receptor.

### DESCRIPTION OF THE FIGURES

FIG. 1 Sensitivity of detection for mutations of E746_A750del and L858R using EGFR Scorpion Kit. (a) Standard DNA with E746_A750del were used at various volumes of 10,000 pg (10⁴), 1,000 pg (10³), 100 pg (10²), 10 pg (10¹) and 1 pg (10⁰). Standard DNA with wild type (Wild) and distilled water (D.W.) were used as negative controls at the same experiment. (b) Standard DNA with E746_A750del at concentrations from 1 pg to 10,000 pg were mixed with 10,000 pg of standard DNA with wild type at a ratio of 1:1 (10⁰), 1:10 (10−1), 1:100 (10⁻²), 1:1,000 (10⁻³) and 1:10,000 (10⁻⁴). (c) Primary curve and 2nd derivative curve represented from standard DNA with E746_A750del at a volume of 10,000 pg. The 2nd derivative represents the rate of change in the slope of the growth curve. The threshold cycle is defined as a cycle number at the highest peak of the 2nd derivative curve (the vertical line in FIG. 1c). (d) Standard curves were derived by plotting the Ct of each curve (shown in FIGS. 1A and 1B) against the log of the standard DNA volume.

FIG. 2 Detection of E746_A750del in genomic DNA derived from lung cancer cell lines. (a) PC-9 with E746_A750del and A431 with wild type. (b) 11_{—}18 with L858R and A431

FIG. 3 Progression free survival (A) and overall survival (B) with respect to the EGFR mutation status of non-small cell lung cancer. (*) Log-rank test.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1 N.Y.), and Gene Transfer and Expression Protocols, pp. 109-128, ed. E. J. Murray, The Humana Press Inc., Clifton, N.J.). These documents are incorporated herein by reference. Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

### Biomarkers

Various biological markers, known as biomarkers, have been identified and studied through the application of biochemistry and molecular biology to medical and toxicological states. Biomarkers can be discovered in both tissues and biofluids, where blood is the most common biofluid used in biomarker studies (Proteomics 2000; 1:1-13, Physiol. 2005; 563:23-60).

A biomarker ran be described as “a characteristic that is objectively measured and evaluated as an indicator of normal biologic processes, pathogenic processes, or pharmacologic responses to a therapeutic intervention”. A biomarker is any identifiable and measurable indicator associated with a particular condition or disease where there is a correlation between the presence or level of the biomarker and some aspect of the condition or disease (including the presence of, the level or changing level of, the type of, the stage of, the susceptibility to the condition or disease, or the responsiveness to a drug used for treating the condition or disease). The correlation may be qualitative, quantitative, or both qualitative and quantitative. Typically a biomarker is a compound, compound fragment or group of compounds. Such compounds may be any compounds found in or produced by an organism, including proteins (and peptides), nucleic acids and other compounds.

Biomarkers may have a predictive power, and as such may be used to predict or detect the presence, level, type or stage of particular conditions or diseases (including the presence or level of particular microorganisms or toxins), the susceptibility (including genetic susceptibility) to particular conditions or diseases, or the response to particular treatments (including drug treatments). It is thought that biomarkers will play an increasingly important role in the future of drug discovery and development, by improving the efficiency of research and development programs. Biomarkers can be used as diagnostic agents, monitors of disease progression, monitors of treatment and predictors of clinical outcome. For example, various biomarker research projects are attempting to identify markers of specific cancers and of specific cardiovascular and immunological diseases.

The term ‘ErbB receptor drug’ used herein includes drugs acting upon the erbB family of receptor tyrosine kinases, which include EGFR, ErbB2 (HER), ErbB3 and ErbB4. In an embodiment the ErbB receptor drug is an ErbB receptor tyrosine kinase inhibitor. In a further embodiment the ErbB receptor drug is an EGFR tyrosine kinase inhibitor. Examples of EGF receptor tyrosine kinase inhibitors include but are not limited to gefitinib, Erlotinib (OSI-774, CP-358774), PKI-166, EKB-569, HKI-272 (WAY-177820), lapatinib (GW2016, GW-572016), canertinib (CI-1033, PD183805), AEE788, XL647, BMS 5599626 or any of the compounds as disclosed in WO2004/006846, WO2003/082831, or WO2003/082290. In particular, gefitinib (also known as Iressa™, by way of the code number ZD1839 and Chemical Abstracts Registry Number 184475-35-2) is disclosed in International Patent Application WO 96/33980 and is a potent inhibitor of the epidermal growth factor receptor (EGFR) family of tyrosine kinase enzymes such as ErbB1.

In another embodiment the ErbB receptor drug is an anti-EGFR antibody such as for example one of cetuximab (C225), matuzumab (EMD-72000), panitumumab (ABX-EGF/rHuMAb-EGFr), MR1-1, IMC-11F8 or EGFRL11. The ErbB receptor drugs mentioned herein may be used as monotherapy or in combination with other drugs of the same or different classes. In a particular embodiment the EGF receptor tyrosine kinase inhibitor is gefitinib.

‘Survival’ encompasses a patients' ‘overall survival’ and ‘progression-free survival’. ‘Overall survival’ (OS) is defined as the time from the initiation of gefitinib administration to death from any cause. ‘Progression-free survival’ (PFS) is defined as the time from the initiation of gefitinib administration to first appearance of progressive disease or death from any cause.

‘Response’ is defined by measurements taken according to ‘Response Evaluation Criteria in Solid Tumours’ (RECIST) involving the classification of patients into two main groups: those that show a partial response or stable disease and those that show signs of progressive disease.

‘Amplification’ reactions are nucleic acid reactions which result in specific amplification of target nucleic acids over non-target nucleic acids. The polymerase chain reaction (PCR) is a well known amplification reaction.

‘Cancer’ is used herein to refer to neoplastic growth arising from cellular transformation to a neoplastic phenotype. Such cellular transformation often involves genetic mutation; in the context of the present invention, transformation involves genetic mutation by alteration of one or more Erb genes as described herein.

The term ‘probe’ refers to single stranded sequence-specific oligonucleotides which have a sequence that is exactly complementary to the target sequence of the allele to be detected.

The term ‘primer’ refers to a single stranded DNA oligonucleotide sequence or specific primer capable of acting as a point of initiation for synthesis of a primer extension product which is complementary to the nucleic acid strand to be copied. The length and sequence of the primer must be such that they are able to prime the synthesis of extension products.

The present application describes -ErbB nucleic acid mutants. As used herein, the term ‘ErbB receptor mutants’ is used to denote a nucleic acid encoding any member of the ErbB family of tyrosine kinase receptors. The term ‘ErbB receptor’ thus encompasses all known human ErbB receptor homologues and variants, as well as other nucleic acid molecules which show sufficient homology to ErbB receptor family members to be identified as ErbB receptor homologues. Preferably, EGFR is identified as a nucleic acid having the sequence for EGFR shown as SEQ ID NO.1.

The term ‘nucleic acid’ includes those polynucleotides capable of hybridising, under stringent hybridisation conditions, to the naturally occurring nucleic acids identified above, or the complement thereof. ‘Stringent hybridisation conditions’ refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at about 65° C.

### Methods for Detection of Nucleic Acids

The detection of mutant nucleic acids encoding ErbB receptors can be employed, in the context of the present invention, to predict the response to drug treatment. Since mutations in ErbB receptor genes generally occur at the DNA level, the methods of the invention can be based on detection of mutations in genomic DNA, as well as transcripts and proteins themselves. It can be desirable to confirm mutations in genomic DNA by analysis of transcripts and/or polypeptides, in order to ensure that the detected mutation is indeed expressed in the subject.

Mutations in genomic nucleic acid are advantageously detected by techniques based on mobility shift in amplified nucleic acid fragments. For instance, Chen et al., Anal Biochem 1996 Jul. 15; 239(1):61-9, describe the detection of single-base mutations by a competitive mobility shift assay. Moreover, assays based on the technique of Marcelino et al., BioTechniques 26(6): 1134-1148 (June 1999) are available commercially.

In a preferred example, capillary heteroduplex analysis may be used to detect the presence of mutations based on mobility shift of duplex nucleic acids in capillary systems as a result of the presence of mismatches.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according. to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods can be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

### Polymerase Chain Reaction (PCR)

PCR is a nucleic acid amplification method described inter alia in U.S. Pat. Nos. 4,683,195 and 4,683,202. PCR consists of repeated cycles of DNA polymerase generated primer extension reactions. The target DNA is heat denatured and two oligonucleotides, which bracket the target sequence on opposite strands of the DNA to be amplified, are hybridised. These oligonucleotides become primers for use with DNA polymerase. The DNA is copied by primer extension to make a second copy of both strands. By repeating the cycle of heat denaturation, primer hybridisation and extension, the target DNA can be amplified a million fold or more in about two to four hours. PCR is a molecular biology tool, which must be used in conjunction with a detection technique to determine the results of amplification. An advantage of PCR is that it increases sensitivity by amplifying the amount of target DNA by 1 million to 1 billion fold in approximately 4 hours. PCR can be used to amplify any known nucleic acid in a diagnostic context (Mok et al., (1994), Gynaecologic Oncology, 52: 247-252).

### Self-Sustained Sequence Replication (3SR)

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42° C.

### Ligation Amplification (LAR/LAS)

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

### Qβ Replicase

In this technique, RNA replicase for the bacteriophage Qβ, which replicates single-stranded RNA, is used to amplify the target DNA, as described by Lizardi et al. (1988) Bio/Technology 6:1197. First, the target DNA is hybridised to a primer including a T7 promoter and a Qβ 5′ sequence region. Using this primer, reverse transcriptase generates a cDNA connecting the primer to its 5′ end in the process. These two steps are similar to the TAS protocol. The resulting heteroduplex is heat denatured. Next, a second primer containing a Qβ 3′ sequence region is used to initiate a second round of cDNA synthesis. This results in a double stranded DNA containing both 5′ and 3′ ends of the Qβ bacteriophage as well as an active T7 RNA polymerase binding site. T7 RNA polymerase then transcribes the double-stranded DNA into new RNA, which mimics the Qβ. After extensive washing to remove any unhybridised probe, the new RNA is eluted from the target and replicated by Qβ replicase. The latter reaction creates 10⁷ fold amplification in approximately 20 minutes.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment.

Each displaced strand is then available to anneal with amplification primers as above. The process continues with repeated nicking, extension and displacement of new DNA strands, resulting in exponential amplification of the original DNA target.

Once the nucleic acid has been amplified, a number of techniques are available for detection of single base pair mutations. One such technique is Single Stranded Conformational Polymorphism (SSCP). SCCP detection is based on the aberrant migration of single stranded mutated DNA compared to reference DNA during electrophoresis. Mutation produces conformational change in single stranded DNA, resulting in mobility shift. Fluorescent SCCP uses fluorescent-labelled primers to aid detection. Reference and mutant DNA are thus amplified using fluorescent labelled primers. The amplified DNA is denatured and snap-cooled to produce single stranded DNA molecules, which are examined by non-denaturing gel electrophoresis.

Chemical mismatch cleavage (CMC) is based on the recognition and cleavage of DNA mismatched base pairs by a combination of hydroxylamine, osmium tetroxide and piperidine. Thus, both reference DNA and mutant DNA are amplified with fluorescent labelled primers. The amplicons are hybridised and then subjected to cleavage using Osmium tetroxide, which binds to an mismatched T base, or Hydroxylamine, which binds to mismatched C base, followed by Piperidine which cleaves at the site of a modified base. Cleaved fragments are then detected by electrophoresis.

Techniques based on restriction fragment polymorphisms (RFLPs) can also be used. Although many single nucleotide polymorphisms (SNPs) do not permit conventional RFLP analysis, primer-induced restriction analysis PCR (PIRA-PCR) can be used to introduce restriction sites using PCR primers in a SNP-dependent manner. Primers for PIRA-PCR which introduce suitable restriction sites can be designed by computational analysis, for example as described in Xiaiyi et al., (2001) Bioinformatics 17:838-839.

Furthermore, techniques based on WAVE analysis can be used (Methods Mol. Med. 2004; 108:173-88). This system of DNA fragment analysis can be used to detect single nucleotide polymorphisms and is based on temperature-modulated liquid chromatography and a high-resolution matrix (Genet Test. 1997-98; 1(3):201-6.)

Real-time PCR (also known as Quantitative PCR, Real-time Quantitative PCR, or RTQ-PCR) is a method of simultaneous DNA quantification and amplification (Expert Rev. Mol. Diagn. 2005(2):209-19). DNA is specifically amplified by polymerase chain reaction. After each round of amplification, the DNA is quantified. Common methods of quantification include the use of fluorescent dyes that intercalate with double-strand DNA and modified DNA oligonucleotides (called probes) that fluoresce when hybridised with a complementary DNA.

Specific primers known as ‘Scorpion® primers’ can be used for a highly sensitive and rapid DNA amplification system. Such primers combine a probe with a specific target sequence in a single molecule, resulting in a fluorescent detection system with unimolecular kinetics (Nucl. Acids Res. 2000; 28:3752-3761). This has an advantage over other fluorescent probe systems such as Molecular Beacons and TaqMan®, in that no separate probe is required to bind to the amplified target, making detection both faster and more efficient. A direct comparison of the three detection methods (Nucl. Acids Res 2000; 28:3752-3761) indicates that Scorpions® perform better than intermolecular probing systems, particularly under rapid cycling conditions. The structure of one version of a Scorpion® primer is such that it is held in a hairpin loop conformation by complementary stem sequences of around six bases which flank a probe sequence specific for the target of interest (Nat. Biotechnol. 1999; 17:804-807). The stem also serves to position together a fluorescent reporter dye (attached to the 5′-end) in close proximity with a quencher molecule. In this conformation, no signal is produced. A PCR-blocker separates the hairpin loop from the primer sequence, which forms the 3′-end of the Scorpion®. The blocker prevents read-through, which would lead to unfolding of the hairpin loop in the absence of a specific target. During PCR, extension occurs as usual from the primer. After the subsequent denaturation and annealing steps, the hairpin loop unfolds and, if the correct product has been amplified, the probe sequence binds to the specific target sequence downstream of the primer on the newly synthesised strand. This new structure is thermodynamically more stable than the original hairpin loop. A fluorescent signal is now generated, since the fluorescent dye is no longer in close proximity to the quencher. The fluorescent signal is directly proportional to the amount of target DNA.

An alternative Scorpion® primer comprises a duplex of two complementary labelled oligonucleotides. One oligonucleotide of the duplex is labelled with a 5′ end reporter dye and carries both the blocker non-coding nucleotide and PCR primer elements, while the other oligonucleotide is labelled with a 3′ end quencher dye. The mechanism of action is then essentially the same as the Scorpion® hairpin primer described above: during real-time quantitative PCR, the 5′ end reporter and 3′ end quencher dyes are separated from each other leading to a significant increase in fluorescence emission.

Scorpions® can be used in combination with the Amplification Refractory Mutation System (ARMS) (Nucl. Acids Res. 1989; 17:2503-2516, Nat. Biotechnol. 1999; 17:804-807) to enable single base mutations to be detected. Under the appropriate PCR conditions a single base mismatch located at the 3′-end of the primer is sufficient for preferential amplification of the perfectly matched allele (Newton et al., 1989), allowing the discrimination of closely related species. The basis of an amplification system using the primers described above is that oligonucleotides with a mismatched 3′-residue will not function as primers in the PCR under appropriate conditions. This amplification system allows genotyping solely by inspection of reaction mixtures after agarose gel electrophoresis. It is simple and reliable and will clearly distinguish heterozygotes at a locus from homozygotes for either allele. ARMS does not require restriction enzyme digestion, allele-specific oligonucleotides as conventionally applied, or the sequence analysis of PCR products.

### EXAMPLE 1

### Clinical Trials and Collection of Blood Serum Samples

The present study was carried out as a correlative study in a multicenter clinical phase II trial for gefitinib monotherapy. The study was conducted with the approval of the appropriate ethical review boards based on the recommendations of the Declaration of Helsinki for biomedical research involving human subjects. Japanese patients with stage IIIB or IV histologically or cytologically proven chemotherapy-naïve NSCLC were enrolled in this trial. Gefitinib was orally administrated to all patients at a fixed dosage of 250 mg daily. Efficacy was assessed using the “Response Evaluation Criteria in Solid Tumours (RECIST)” guidelines (J. Natl. Cancer Inst. 2000; 92:205-216).

Twenty-eight patients were enrolled between Oct. 23, 2002, to Aug. 3, 2003 (Table 1). All patients were evaluated for response and followed for progression free survival and overall survival. Blood samples (2 ml) from 27 patients were collected before the initiation of gefitinib administration. Serum DNA was extracted in all 27 samples at concentrations of up to 1720 ng/ml.

Sample collection and DNA extraction. Blood samples from the 26 NSCLC patients were collected before the initiation of gefitinib administration. Separated serum was stocked at −80° C. until use. Serum DNA was extracted and purified using Qiamp Blood Kit (Qiagen, Hilden, Germany), with the following protocol modifications. One column was used repeatedly until the whole sample had been processed. The resulting DNA was eluted in 50 μl of sterile bidistilled buffer. The concentration and purity of the extracted DNA were determined by spectrophotometry. The extracted DNA was stocked at −20° C. until use.

### EXAMPLE 2

### Use of Scorpion Primers and the Amplification Refractory Mutation System (ARMS) to Detect E746 A750 del and L858R EGFR Mutations

### Sensitivities of EGFR Scorpion® Kit

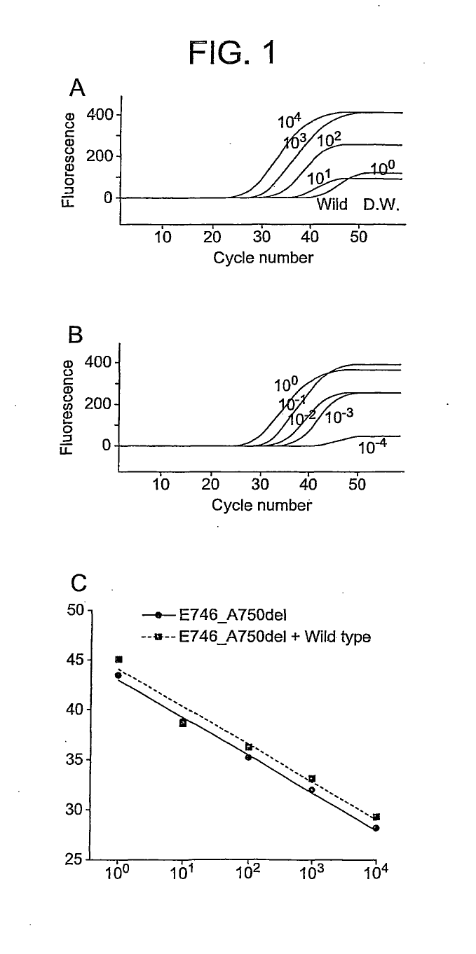

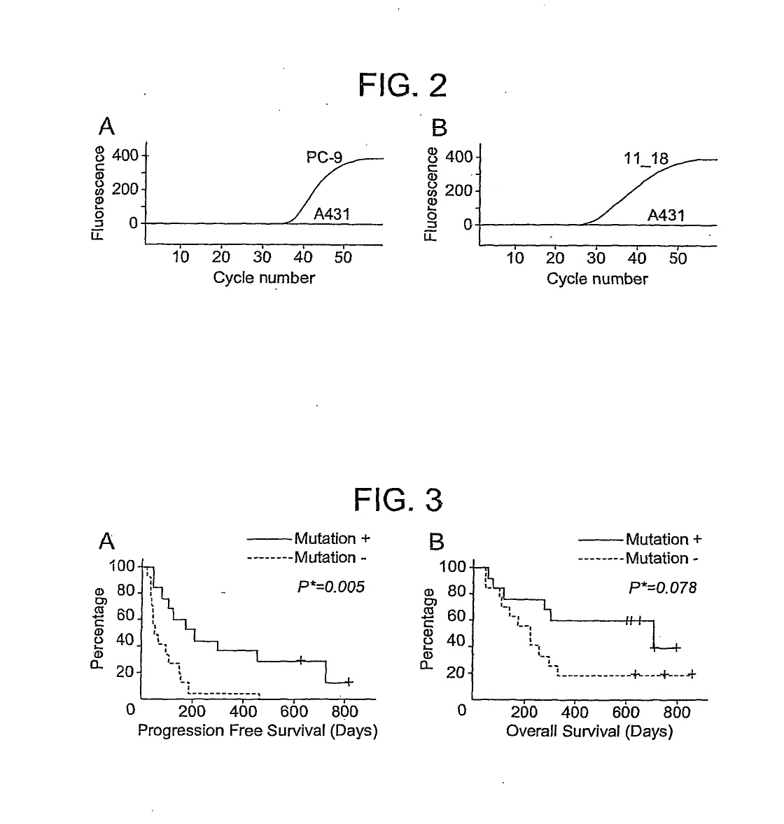

Preliminary experiments are performed to evaluate the sensitivities of EGFR Scorpion Kit (FIG. 1). All curves using E746_A750del standard DNA at a volume from 1 pg to 10,000 pg were increased by reaching up to 45 cycles (FIG. 1a). When wild standard DNA and water were used as negative controls, the curves were not increased and continued flat at reaching to 50 cycles (FIG. 1a). Using diluted E746_A750del standard DNA with wild standard DNA at ratio from 10⁰ to 10⁻⁵, all curves which indicated the presence of E746_A750del were increased by reaching up to 45 cycles (FIG. 1b). Standard curves in the range of measured volumes in this study were linear with r² values of 0.997 and 0.987. Both slopes of curves were almost parallel (FIG. 1c). Ct of diluted E746_A750del standard DNA with wild DNA was close to that of only E746_A750del standard DNA in every volume of E746_A750del standard DNA. Although peak fluorescence level of diluted E746_A750del standard DNA with wild DNA was lower than without wild DNA standard at ratio less than 10⁻³, the presence of E746_A750del were clearly detected. The curves of DNA with E746_A750del at an amount of up to 1 pg were unaffected by interfusion of DNA of wild type EGFR. In the cell based experiments using genomic DNA of human cancer cell lines, the signal using DNA derived from the PC-9 cells was detected and that from the A431 cells was not detected as expected (FIG. 2).

We used EGFR Scorpion™ Kit (DxS Ltd, Manchester, UK) which combined two technologies, namely ARMS™ and Scorpion™, to detect mutations in real time PCR reactions. Four kinds of scorpion primers for detections of E746_A750del, L858R and wild type in both exon 19 and exon 21 were designed and synthesized by DxS Ltd (Manchester, UK). The sequences of the scorpion primer for E746_A750 del and L858R were based on the GenBank-archived human sequence for EGFR (accession number: AY588246). All reactions were performed in 25 μl volumes using 1 μl of template DNA, 7.5 μl of Reaction buffer mix, 0.6 ml of Primer mix and 0.1 ml of Taq polymerase. All regents are included in this kit. Real time PCR were carried out using SmartCycler® II (Cepheid, Sunnyvale, Calif.) in the following conditions which were initial denaturation at 95° C. for 10 minutes, 50 cycles of 95° C. for 30 seconds, 62° C. for 60 seconds with fluorescence reading (set to FAM that allows optical excitation at 480 nm and measurement at 520 nm) at the end of each cycle. Data analysis was performed with Cepheid SmartCycler software (Ver. 1.2b). The threshold cycle (Ct) was defined as the cycle at the highest peak of the 2nd derivative curve, which represented the point of maximum curvature of the growth curve. Both Ct and maximum fluorescence (Fl) were used for interpretation of the results. Positive results were defined as follows: Ct≦45 and Fl≧50. These analyses were performed in duplicate for each sample. To confirm the sensitivities for the detection of E746_A750del, we used the standard DNA which was included in EGFR Scorpion Kit. Standard DNA with E746_A750del at a volume of 1, 10, 100, 1,000 or 10,000 pg, and the mixture of standard DNA with wild type at 10,000 pg and standard DNA with E746_A750del at a volume of 1, 10, 100, 1,000 or 10,000 pg were used. For quantification, a standard curve was generated by plotting the cycle number of Ct against the log of the DNA volume of the known standards. The linear correlation coefficient (R²) values and the formula of the slopes were calculated. DNA for the positive control were extracted from a Japanese human adenocarcinoma PC-9 cell line known to contain E746_A750del and a human epidermoid carcinoma A431 cell line known to contain a wild type in exon 19 and 10,000 pg of their DNA were used.

### EGFR Mutation Status of Serum DNA Detected by ARMS

E746_A750del or L858R of serum DNA derived from twenty-seven NSCLC patients was examined. Wild type in both exon 19 and exon 21 were detected from all serum samples. E746_A750del was detected in samples of 12 patients. L858R was detected in one patient (Table 2). Totally, EGFR mutations were detected in 13 out of 27 (48.1%) patients. The histological subtypes of original tumours were summarised in Table 3a in the 23 patients with the EGFR mutation in serum. The 11 out of 23 (47.8%) cases of adenocarcinoma, 1 out of 2 cases of squamous cell carcinoma, and 1 out of 2 cases of large cell carcinoma were positive for EGFR mutations. EGFR mutation status was not correlated statistically with histogocal type. EGFR mutation was more frequently detected in the samples derived from women patients than those of men (7 of 10; 70% vs 6 of 17; 29.4%, Table 3b).

EGFR Mutation Status in Serum and Response to gefitinib

The EGFR mutation was significantly more frequently observed in the samples from the patients who showed a partial response (PR) or stable disease (SD) (11 out of 17 cases, 75%) than in samples from patients with progressive disease (PD, 2 out of 10 cases, 18%) (p=0.046, Fisher's exact test, Table 3c).

### EXAMPLE 3

### EGFR Mutation Status in Serum and Impact on Survival

Statistical analysis. Fisher's exact test was used to compare the presence of EGFR mutations in NSCLC patients with different characteristics, including gender, tumour type and response to gefitinib. Regarding analyses of response to gefitinib, patients were categorised into two groups of partial response or stable disease (PR/SD) and progressive disease (PD) depending on the RECIST criteria. We compared Kaplan-Meier curves for overall survival and progression-free survival using the standard log-rank test. Overall survival (OS) was defined as the time from the initiation of gefitinib administration to death from any cause; patients known to be still alive at the time of the analysis were censored at the time of their last follow-up. Progression-free survival (PFS) was defined as the time from the initiation of gefitinib administration to first appearance of progressive disease or death from any cause; patients known to be alive and without progressive disease at the time of analysis were censored at the time of their last follow-up. A P value of 0.05 was considered to be statistically significant. The statistical analyses were performed using the Stat View software package, version 5.0.

Median PFS of all of the patients treated with gefitinib was 98 days and median OS was 306 days. The patients with EGFR mutations in serum showed significantly longer median PFS compared with the patients without EGFR mutations (200 days v 46 days, P=0.005, FIG. 3a). The patients with EGFR mutations showed longer median OS compared with the patients without EGFR mutations, although there was no statistical significance (611 days v 232 days, P=0.078, FIG. 3b). These results suggest that serum EGFR mutation behaves as an prognostic factor for progression free survival and overall survival as well as a predictor of response in the patients treated with gefitinib.

### EXAMPLE 4

### EGFR Mutation in Serum Analysed by Direct Sequence and in Comparison with ARMS

The deletional mutation (E746_A750del) was detected by direct sequence in serum DNA extracted from 10 out of 27 patients (37.0%).

PCR amplification and direct sequencing. Amplification and direct sequencing were performed in duplicate for each sample obtained from serum and tissue specimen. PCR was performed in 25 μl volumes using 15 μl of template DNA, 0.75 units of Ampli Taq Gold DNA polymerase (Perkin-Elmer, Roche Molecular Systems, Inc., Branchburg, N.J.), 2.5 μl of PCR buffer, 0.8 mM dNTP, 0.5 μM of each primer, and different concentrations of MgCl₂, depending on the polymorphic marker. The sequences of primer sets and schedules of amplifications were followed as described previously (Nuc. Acids Res. 1989; 17:2503-2516). The amplification was performed using a thermal cycler (Perkin-Elmer, Foster City, Calif.). Sequencing were performed using an ABI prism 310 (Applied Biosystems, Foster City, Calif.). The sequences were compared with the GenBank-archived human sequence for EGFR (accession number: AY588246).

No point mutation in exons 18, 19, 20 and 21 was detected in the PCR products from serum samples. The serum EGFR status detected by direct sequence was not correlated statistically with neither the histological type, the gender, the responsiveness of gefitinib (Table 3), and the survival benefit (PFS: P=0.277, OS: P=0.859, suppl data 2). The EGFR mutation status by direct sequence was consistent with those by ARMS in 15/27 (55.6%) of the paired samples. EGFR mutations (E746_A750del) in four cases were positive by direct sequence and negative by ARMS. Eight cases were negative by direct sequence and positive by ARMS.

### EXAMPLE 5

### EGFR Mutations in Tumours in Comparison with Those in Serum

Twenty tumour samples were obtained from the 15 patients retrospectively.

Tissue sample collection and DNA extraction. Tumour specimens were obtained on protocols approved by the Institutional Review Board. Twenty paraffin blocks of tumour material, obtained from 15 patients for diagnoses before treatment, were collected retrospectively. 11 tumour samples were collected from primary cancer via trans bronchial lung biopsy, 1 was resected by operation, 9 were from metastatic sites (4 from bone, 3 lymph nod, 1 brain and 1 colon). All specimens underwent histological examination to confirm the diagnosis of NSCLC. DNA extraction from tumour samples was performed using DEXPAT™ kit (TaKaRa Biomedicals, Shiga, Japan).

Sequencing of exons 19 and 21 in EGFR were performed under the same PCR conditions. The tumour samples from 12 patients were sequenced (Table 4). EGFR mutations were detected in 4 cases (25.0%); Three of them were 15 bp deletion (E746_A750del) in exon 19 and one case was L858R in exon 21. Histological type of patients with EGFR mutations were adenocarcinoma in 3 and large cell carcinoma in 1. The responses to gefitinib in these four patients were PR in 2, SD in 1, and PD in 1 patient. Other three samples were not evaluated because of low amplification of PCR products.

Pairs of tumour samples and serum samples were obtained from 11 patients retrospectively (Table 4). The EGFR mutation status in the tumours was consistent with those in serum of 8/11 (72.7%) in the paired samples. The E746_A750del was positive in the tumour and negative in the serum in two patients, and the E746_A750del was negative in the tumour and positive in the serum in a patient.

TABLE 1
Patient characteristics

| | (n) |
|---|---|
| No. of. Patients | 28 |

Age (years)

| | |
|---|---|
| Median | 64 |
| Range | 44-87 |

Sex

| | |
|---|---|
| Male | 18 |
| Female | 10 |

PS

| | |
|---|---|
| 0 | 19 |
| 1 | 7 |
| 2 | 2 |

Stage

| | |
|---|---|
| IIIB | 3 |
| IV | 25 |

Histology

| | |
|---|---|
| Ad | 23 |
| Scc | 2 |
| Large | 2 |

Response

| | |
|---|---|
| PR | 9 |
| SD | 8 |
| PD | 11 |

PS, performance status; Ad, adenocarcinoma; Scc, squamous cell carcinoma; Large, large cell carcinoma; PR, partial response; SD, stable disease; PD, progressive disease.

TABLE 2
Patients' Characteristics and EGFR Mutant Status Detected
from Serum DNA Using EGFR ARMS-Scorpion Method

| | | | Exon 19 | | Exon 21 | |
|---|---|---|---|---|---|---|
| Response | Gender | Histology | Wild | E746_A750deI | Wild | L858R |
| PR | M | Ad | + | − | + | + |
| PR | F | Ad | + | + | + | − |
| PR | M | Ad | + | − | + | − |
| PR | F | Ad | + | + | + | − |
| PR | M | Ad | + | + | + | − |
| PR | F | Ad | + | − | + | − |
| PR | M | Ad | + | + | + | − |
| PR | F | Ad | + | + | + | − |
| PR | F | Ad | + | + | + | − |
| SD | M | Large | + | − | + | − |
| SD | F | Ad | + | + | + | − |
| SD | M | Ad | + | − | + | − |
| SD | F | Ad | + | − | + | − |
| SD | F | Ad | + | + | + | − |
| SD | M | Ad | + | − | + | − |
| SD | F | Ad | + | + | + | − |
| SD | M | SCC | + | + | + | − |
| PD | F | Scc | + | − | + | − |
| PD | M | Ad | + | − | + | − |
| PD | M | Ad | + | − | + | − |
| PD | M | Large | + | + | + | − |
| PD | M | Ad | + | − | + | − |
| PD | M | Ad | + | − | + | − |
| PD | M | Ad | + | − | + | − |
| PD | M | Ad | + | − | + | − |
| PD | M | Ad | + | + | + | − |
| PD | M | Ad | + | − | + | − |

SD, stable disease; PD, progressive disease; PR, partial response; M, male; F, female; Ad, adenocarcinoma; Large, large cell carcinoma; Scc, squamous cell carcinoma; +, Curve detected by SmartCycler; −, Curve not detected by SmartCycler;

TABLE 3
Frequency of EGFR mutations in serum DNA from lung
cancer patients according to histology (a), gender
(b), and response to gefitinib (c). Total 27 samples
were obtained from 28 patients before treatment.

| | |
|---|---|
| EGFR Scorpion Kit | Direct sequence |

| | | | |
|---|---|---|---|
| + | − | + | − |

a Histology and EGFR Mutant States

| | | | | | | |
|---|---|---|---|---|---|---|
| Ad | 11 | 12 | | 8 | 15 | |
| Non Ad | 2 | 2 | P > 0.999 | 2 | 2 | P > 0.999 |

b Gender and EGFR Mutant States

| | | | | | | |
|---|---|---|---|---|---|---|
| Female | 7 | 3 | | 5 | 5 | |
| Male | 6 | 11 | P = 0.120 | 5 | 12 | P = 0.415 |

c Response to gefitinib and EGFR Mutant States

| | | | | | | |
|---|---|---|---|---|---|---|
| PR/SD | 11 | 6 | | 8 | 9 | |
| PD | 2 | 8 | P = 0.046 | 2 | 8 | P = 0.231 |

Ad, adenocarcinoma PR, partial response; SD, stable disease; PD, progressive disease;

TABLE 4
EGFR mutation status in tumour samples and serum samples. Pairs of both
tumour samples and serum samples were obtained from 12 patients.

| | | | EGFR mutation status | | | | |
|---|---|---|---|---|---|---|---|
| | | | | EGFR Scorpion Kit | | | |
| | | | | Exon 19 | | Exon 21 | |
| Gender | Histology | Response | Tumour sample | Wild | Mutation | Wild | Mutation |
| M | Large | SD | Wild | + | − | + | − |
| F | SCC | PD | Wild | + | − | + | − |
| M | Adeno | PD | Wild | + | − | + | − |
| M | Adeno | PR | L858R | + | − | + | + |
| F | Adeno | SD | Wild * | + | + | + | − |
| M | Large | PD | E746-A750 del | + | + | + | − |
| M | Adeno | PD | Wild | + | − | + | − |
| M | Adeno | PD | Wild | + | − | + | − |
| M | Adeno | SD | E746-A750 del * | + | − | + | − |
| F | Adeno | PR | E746-A750 del * | + | − | + | − |
| M | Adeno | PD | Wild | + | − | + | − |

M, male; F, female; SD, stable disease; PD, progressive disease; PR, partial response; Scc, squamous cell carcinoma; Ad, adenocarcinoma; Large, large cell carcinoma
* patients who have different states of EGFR mutation from tumour-derived DNA and serum- derived DNA.

TABLE 5

| | | |
|---|---|---|
| Position | Wild type | Mutant |

Protein

| | | |
|---|---|---|
| 688 | L | P |
| 694 | P | L/S |
| 709 | E | K |
| 709 | E | V |
| 715 | I | S |
| 720 | S | F |
| 718 | L | P |
| 719 | G | S/C/A/D |
| 724 | G | S |
| 730 | L | F |
| 733 | P | L |
| 735 | G | S |
| 742 | V | A |
| delE746_A750 | | |
| delE746_S752V | | |
| delE746_P753insLS | | |
| delL747_A750insP | | |
| delL747_T751insP | | |
| 746 | E | K |
| del750_754 | | |
| 751 | T | I |
| 752 | S | Y |
| 755 | A | P |
| del756_758 | | |
| 761 | D | N |
| 768 | S | I |
| 769 | V | L |
| 770 | D | N |
| 772 | H | L |
| 772 | P | S |
| 773 | V | M |
| 776 | R | C |
| 778 | G | F |
| 781 | C | R |
| 783 | T | I |
| 784 | S | F |
| 790 | T | M |
| 792 | L | P |
| 798 | L | F |
| 810 | G | S |
| 820 | Q | STOP |
| 826 | N | S |
| 834 | V | M |
| 835 | H | Y |
| 836 | R | C |
| 847 | T | I |
| 850 | H | N |
| 851 | V | A |
| 853 | I | T |
| 857 | G | R |
| 858 | L | M |
| 858 | L | R |
| 859 | A | T |
| 861 | L | Q |
| 863 | G | D |
| 864 | A | T/V |
| 866 | E | K |
| 873 | G | E |
| 877 | P | S |
| 880 | W | STOP |
| 882 | A | T |
| 893 | H | Q |
| 895 | S | G |
| 897 | V | I |
| 958 | R | P |
| Nucleotide | | |
| 2063 | C | T |
| 2080 | C | T |
| 2081 | C | T |
| 2118 | C | T |
| 2125 | G | A |
| 2126 | A | T |
| 2142 | G | A |
| 2144 | T | G |
| 2153 | T | C |
| 2155 | G | T/A/C |
| 2156 | G | C/A |
| 2159 | C | T |
| 2169 | C | T |
| 2170 | G | A |
| 2188 | C | T |
| 2198 | C | T |
| 2203 | G | A |
| 2225 | T | C |
| 2236 | G | A |
| del2247_2262 | | |
| 2252 | C | T |
| del2268_2275 | | |
| 2281 | G | A |
| 2303 | T | G |
| 2305 | G | C |
| 2308 | G | A |
| 2314 | C | T |
| 2326 | C | T |
| 2340 | C | T |
| 2341 | T | C |
| 2348 | C | T |
| 2351 | C | T |
| 2364 | C | T |
| 2369 | C | T |
| 2375 | T | C |
| 2392 | C | T |
| 2406 | C | T |
| 2428 | G | A |
| 2421 | C | T |
| 2458 | C | T |
| 2477 | A | G |
| 2484 | G | A |
| 2500 | G | A |
| 2502 | G | A |
| 2503 | C | T |
| 2506 | C | T |
| 2508 | C | T |
| 2523 | G | A |
| 2540 | C | T |
| 2548 | C | A |
| 2552 | T | C |
| 2553 | C | T |
| 2563 | A | T |
| 2565 | G | A |
| 2569 | G | A |
| 2570 | G | T |
| 2571 | G | T |
| 2572 | C | A |
| 2575 | G | A |
| 2582 | T | A |
| 2588 | G | A |
| 2588 | T | C |
| 2590 | G | A |
| 2591 | C | T |
| 2596 | G | A |
| 2607 | C | T |
| 2618 | G | A |
| 2629 | C | T |
| 2639 | G | A |
| 2644 | G | A |
| 2676 | C | T |
| 2679 | C | A |
| 2683 | A | G |
| 2689 | G | A |
| 2877 | A | G |

1. A method for detecting one or more mutations in an ErbB receptor for predicting the response of a patient to an ErbB receptor drug comprising the steps of:
(a) providing a bio-fluid sample from a patient;
(b) extracting DNA from said sample; and
(c) screening said DNA for the presence of one or more mutations in the receptor.

2. A method according to claim 1 for monitoring the response of a patient to an ErbB receptor drug comprising the steps of:
(a) providing a bio-fluid sample from a patient;
(b) extracting DNA from said sample; and
(c) screening said DNA for the presence of one or more mutations in the receptor.

3. A method according to claim 1 comprising detection of one or more mutations in an ErbB receptor that alter the tyrosine kinase activity in said receptor.

4. A method according to a claim 1 wherein the ErbB receptor is EGFR.

5. A method according to claim 1, wherein the prediction of the response of a cancer patient to an ErbB receptor drug predicts the survival benefit to the patient.

6. A method according to claim 1, further comprising the step of:
(d) concluding that patients in which both mutated and wildtype alleles are detected will respond positively to an ErbB receptor drug, whereas patients in which only wild type alleles are detected will not respond positively to the drug.

7. The method of claim 1 wherein the method of screening comprises use of polymerase chain reaction with allele specific primers that detect single base mutations, small in-frame deletions or base substitutions.

8. The method of claim 7 wherein the method of screening involves use of real time polymerase chain reaction (real time-PCR) with allele specific primers that detect single base mutations, small in-frame deletions or base substitutions.

9. The method of claim 7 or 8 wherein a first primer pair is used to detect the wild type allele and a second primer pair is used to detect the mutant allele; and wherein one primer of each pair comprises:
(a) a primer with a terminal 3′ nucleotide that is allele specific for a particular mutation; and
(b) possible additional mismatches at the 3′ end of the primer.

10. The method of claim 9 wherein one primer of each pair comprises:
a single molecule or nucleic acid duplex probe containing both a primer sequence and a further sequence specific for the target sequence;
a fluorescent reporter dye attached to the 5′ end of the probe in close proximity with a quencher molecule within said single molecule or nucleic acid duplex;
one or more non-coding nucleotide residues at one end of said probe;
wherein said reporter dye and quencher molecule become separated during amplification of the target sequence.

11. The method according to claim 10, wherein the probe is a Scorpion® probe.

12. The method claim 1 wherein the mutation is detected using a technique capable of detecting a mutant sequence present at 10% of the level of wild type sequence.

13. The method of claim 1 wherein the bio-fluid is any one of blood, serum, plasma, sweat or saliva.

14. The method of claim 13 wherein the bio-fluid is serum.

15. The method of claim 1 wherein the ErbB receptor drug is an ErbB receptor tyrosine kinase inhibitor.

16. The method of claim 1 wherein the ErbB receptor drug is an EGFR tyrosine kinase inhibitor.

17. The method of claim 15 wherein the drug is selected from a group consisting of gefitinib, erlotinib (Tarceva, OSI-774, CP-358774), PKI-166, EKB-569, HKI-272 (WAY-177820), lapatinib (GW2016, GW-572016, GSK572016), canertinib (CI-1033, PD183805), AEE788, XL647, BMS 5599626, ZD6474 (Zactima™) or any of the compounds as disclosed in WO2004/006846 or WO2003/082290.

18. The method of claim 15 wherein the EGFR tyrosine kinase inhibitor is gefitinib or erlotinib.

19. The method claim 1 wherein the ErbB receptor drug is an anti-EGFR antibody selected from the group consisting of cetuximab (Erbitux, C225), matuzumab (EMD-72000), panitumumab (ABX-EGF/rHuMAb-EGFR), MR1-1, IMC-11F8 or EGFRL11.

20. The method of claim 1 wherein the ErbB receptor drug is used as monotherapy or in combination with other drugs.

21. The method of claim 1 wherein the mutations are insertions, deletions or substitutions of nucleic acid.

22. The method of claim 21 wherein the mutations occur in the tyrosine kinase domain of an ErbB receptor.

23. The method of claim 1 wherein the mutations occur in the tyrosine kinase domain of EGFR.

24. The method of claim 21 wherein the mutations cluster around the ATP binding site in exons 18, 19, 20 or 21 of EGFR.

25. The method of claim 21 wherein the mutations are selected from the group of EGFR mutations listed in Table 5.

26. The method of claim 23 wherein the mutations are E746_A750del in exon 19 and L858R in exon 21 of EGFR.

27. The method of claim 1 wherein the patient suffers from a cancer selected from the group consisting of non-solid tumours such as leukaemia, multiple myeloma or lymphoma, and also solid tumours, for example bile duct, bone, bladder, brain/CNS, glioblastoma, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval tumours.

28. The method of claim 1 further comprising the step of:
(d) screening said DNA for the presence of one or more mutations in components of the downstream signalling pathway of an ErbB receptor.

29. A composition comprising a first primer pair which is used to detect the wild type allele and a second primer pair which is used to detect the mutant allele of an ErbB receptor wherein one primer of each pair further comprises:
(a) a primer with a terminal 3′ nucleotide that is allele specific for a particular mutation; and
(b) possible additional mismatches at the 3′ end of the primer;
(c) a single molecule or nucleic acid duplex probe containing both a primer sequence and a further sequence specific for the target sequence;
(d) a fluorescent reporter dye attached to the 5′ end in close proximity with a quencher molecule within said single molecule or nucleic acid duplex;
(e) one or more non-coding nucleotide residues at one end of said probe;
(f) wherein said reporter dye and quencher molecule become separated during amplification of the target sequence.

30. Use of a primer specific for an ErbB receptor in an assay conducted with a bio-fluid for predicting the response of a patient to an ErbB drug.

31. Use of a primer specific for an ErbB receptor in the manufacture of a composition for testing a bio-fluid for predicting the response of a patient to an ErbB drug.

32. The use according to claim 30 further comprising the steps of:
(a) extracting DNA from said sample; and
(b) screening said DNA for the presence of one or more mutations that alter tyrosine kinase activity in an ErbB receptor.


SEQUENCE LISTING (2 sequences)

SEQ ID NO: 1 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - protein, 1210 aa, Homo sapiens [sequence omitted]